(19) United States
(12) Patent Application Publication
(10) Pub. No.: US 2010/0267041 A1
(43) Pub. Date: Oct. 21, 2010

(54) SERIAL ANALYSIS OF BIOMARKERS FOR DISEASE DIAGNOSIS

(75) Inventors: Anthony P. Shuber, Mendon, MA (US); Cecilia Fernandez, Jamaica Plain, MA (US)

Correspondence Address: BROWN RUDNICK LLP, ONE FINANCIAL CENTER, BOSTON, MA, 02111, US

(73) Assignee: Predictive Biosciences, Inc., Lexington, MA (US)

(21) Appl. No.: 12/774,303

(22) Filed: May 5, 2010

Related U.S. Application Data
(63) Continuation-in-part of application No. 12/034,698, filed on Feb. 21, 2008.
(60) Provisional application No. 60/972,507, filed on Sep. 14, 2007.

(51) Int. Cl. C12Q 1/68

(52) U.S. Cl. 435/6

(57) ABSTRACT

The present invention generally relates to serial analysis of biomarkers for disease diagnosis. In certain embodiments, the invention provides methods for diagnosing a disease including obtaining a sample from a subject, conducting a first assay to determine whether a first biomarker in the sample is positive or negative for a disease, and conducting a second assay to determine whether a second biomarker in the sample is positive or negative for the disease if the first assay produced a negative result.

### RELATED APPLICATION

The present application is a continuation-in-part of U.S. nonprovisional patent application Ser. No. 12/034,698, filed Feb. 21, 2008, which claims the benefit of and priority to U.S. provisional patent application Ser. No. 60/972,507, filed Sep. 14, 2007, the content of each of which is incorporated by reference herein in its entirety.

### FIELD OF THE INVENTION

The present invention generally relates to serial analysis of biomarkers for disease diagnosis.

### BACKGROUND

Biomarkers refer to cellular, biochemical, molecular or genetic alterations by which a normal, abnormal or simply biologic process may be recognized or monitored. Biomarkers are measurable in biological media, such as human tissues, cells or fluids, and may be used to identify pathological processes before individuals become symptomatic or to identify individuals who are susceptible to diseases, such as cancer.

Standard screening assays have been developed that use biomarkers to assess the health status of a patient and to provide insight into the patient's risk of having a particular disease or condition. Screening assays generally employ a threshold above which a patient is screened as “positive” for the indicated disease and below which the patient is screened as “negative” for the indicated disease. Those tests vary not only in accuracy, precision and reliability, but have performance characteristics, e.g., sensitivity, specificity, positive predictive value (PPV) and negative predictive value (NPV). Test sensitivity and specificity refer to the identification of patients with and without the disease, respectively. For a test to be useful, it must have high sensitivity and specificity. The PPV refers to the proportion of persons who tested positive who have the disease, and the NPV refers to the number of persons who tested negative for a disease and who do not have the disease.

A problem with those diagnostic tests used in clinical practice is that the tests are single analyte, i.e., the tests assay only a single biomarker. By looking at only a single biomarker, those tests have limited sensitivity and specificity, and thus a certain number of patients will have a result that does not allow them to be unambiguously placed into any clinical category. Thus, there is always a population of patients (false negatives and false positives) who are referred for improper follow-up due to the ambiguity inherent in the diagnostic test. A false positive can lead to unnecessary diagnostic procedures, including biopsies, and can result in prohibitive costs. Alternatively, a false negative leads to a missed diagnosis and a patient's disease goes untreated.

There is a need for methods that can eliminate as many ambiguous results as possible, thereby limiting the number of patients who must endure unnecessary procedures and optimizing identification of patients who would certainly benefit from continual monitoring and/or intervention.

### SUMMARY

The invention generally relates to methods for assessing the clinical status of a patient. Methods of the invention take advantage of the fact that multiple may be associated with a single disease. In practice, methods of the invention involve a step-wise linear application of different biomarkers, in which a test result of a first biomarker (i.e., positive or negative for a disease) informs whether an additional biomarker should be assayed to gain further information with respect to a disease state in a patient. Using this approach, biomarkers are applied in sequence. A first biomarker is applied to determine whether patients are above or below a cutoff for a particular disease. Then, a second marker is applied to only the population of patients that were below the cutoff of the first biomarker, thus maximizing sensitivity of the assay. The process is iterative and may be repeated as many times as necessary using as many biomarkers as necessary to acquire the needed clinical information regarding a disease state in a patient. Thus methods of the invention achieve greater clinical performance of near 100% PPV and NPV.

In certain aspects, methods of the invention involve obtaining a sample from a subject, conducting a first assay to determine whether a first biomarker in the sample is positive or negative for a disease, and conducting a second assay to determine whether a second biomarker in the sample is positive or negative for the disease if the first assay produced a negative result with respect to the first biomarker. Based on the result of the second biomarker, methods of the invention further include conducting at least one additional assay on the sample, in which the additional assays are conducted serially and each assay is conducted on a different biomarker. The assays are conducted until a positive result with respect to a biomarker indicative of the disease is detected.

The biomarkers used may be any biomarkers known in the art to have a predictive value or suspected predictive value associated with a condition or conditions being diagnosed. Thus the biomarker used will depend on the disease to be diagnosed. In certain embodiments, nucleic acid biomarkers are assayed before protein biomarkers. When the biomarker is a nucleic acid biomarker, the assay may be used to detect presence or absence of a mutation, in which presence of the mutation is indicative of a positive result for the disease. When the biomarker is a protein biomarker, the assay measures a level of the protein in the sample. In certain embodiments, a level exceeding a predetermined threshold for the protein is indicative of a positive result for the disease. In other embodiments, a level below a predetermined threshold for the protein is indicative of a positive result for the disease. The biomarkers have a known standard-of-care threshold for disease diagnosis, which is easily knowable by one of skill in the art by reference to literature.

Another aspect of the invention provides methods that iteratively analyze sets of biomarkers. By including multiple biomarkers at each step, the diagnostic power and accuracy of the result is increased. Those methods of the invention provide for diagnosing a disease including obtaining a sample from a subject, conducting a first set of assays on a first set of biomarkers in the sample, assigning a value for each of the biomarkers in the first set based upon a standard-of-care threshold for disease diagnosis for each biomarker in the first set, aggregating the values into a first single output score, in which the first score is indicative of a positive or a negative diagnosis of a disease, conducting a second set of assays on a second set of biomarkers in the sample if the first single output score is indicative of a negative diagnosis of the disease, assigning a value for each of the biomarkers in the second set based upon a standard-of-care threshold for disease diagnosis for each biomarker in the second set, and aggregating the outputs into a second single output score, in which the second score is indicative of a positive or a negative diagnosis of the disease.

Based on the result of the second set of biomarkers, methods of the invention further include conducting at least one additional set of assays on the sample, in which the additional sets of assays are conducted serially and each set of assays includes a different set of biomarkers. The assays are conducted until an aggregated single output score with respect to a set of biomarkers is obtained that is a positive result for the disease to be diagnosed.

In certain embodiments, the each biomarker in the set is assigned a binary score (e.g., 1/0 or yes/no). In certain embodiments, nucleic acid biomarkers are assayed before protein biomarkers. When the biomarker is a nucleic acid biomarker, the assay may be used to detect presence or absence of a mutation, in which presence of a mutation for a biomarker in the first set is assigned an output of “1”, and absence of a mutation for a biomarker in the first set is assigned an output of “0”.

When the biomarker is a protein biomarker, the assay measures a level of the protein in the sample. In certain embodiments, a level exceeding a predetermined threshold for each protein in the second set is assigned an output of “1”, and a level below a predetermined threshold for each protein in the second set is assigned an output of “0”. In other embodiments, a level below a predetermined threshold for each protein in the second set is assigned an output of “1”, and a level exceeding a predetermined threshold for each protein in the second set is assigned an output of “0”.

### DETAILED DESCRIPTION

The present invention generally relates to serial analysis of biomarkers for disease diagnosis. In certain aspects, methods of the invention involve obtaining a sample from a subject, conducting a first assay to determine whether a first biomarker in the sample is positive or negative for a disease, and conducting a second assay to determine whether a second biomarker in the sample is positive or negative for the disease if the first assay produced a negative result with respect to the first biomarker.

Based on the result of the second biomarker, methods of the invention further include conducting at least one additional assay on the sample, in which the additional assays are conducted serially and each assay is conducted on a different biomarker. The assays are conducted until a positive result with respect to a biomarker indicative of the disease is detected.

A sample can be from a mammal, e.g. a human tissue or body fluid. A tissue is a mass of connected cells and/or extracellular matrix material, e.g. skin tissue, nasal passage tissue, CNS tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, phlegm, saliva, sweat, amniotic fluid, mammary fluid, and cerebrospinal fluid (CSF), such as lumbar or ventricular CSF. A sample may also be a fine needle aspirate or biopsied tissue. A sample also may be media containing cells or biological material. The patient sample from which a biomarker is obtained is immaterial to the functioning of the invention. Preferred sample sources include blood, serum, sputum, stool, saliva, urine, cerebral spinal fluid, breast nipple aspirate, and pus.

Nucleic acids or proteins are extracted from the sample according to methods known in the art. See for example, Maniatis, et al., Molecular Cloning: A Laboratory Manual, Cold Spring Harbor, N.Y., 1982, the content of which is incorporated by reference herein in its entirety.

Biomarkers chosen are immaterial to the operation of the invention as long as the marker is associated with the disease for which screening is being conducted. Exemplary biomarkers include nucleic acid biomarkers and protein biomarkers. Biomarkers used in methods of the invention are chosen based upon their predictive value or suspected predictive value for the condition or conditions being diagnosed. Particular markers are selected based upon various diagnostic criteria, such as suspected association with disease. The number of markers chosen will depend on the number of assays performed and is at the discretion of the user. Biomarkers should be chosen that cumulatively increase the specificity/sensitivity of the assay. A panel of markers can be chosen to increase the effectiveness of diagnosis, prognosis, treatment response, and/or recurrence. In addition to general concerns around specificity and sensitivity, markers can also be chosen in consideration of the patient's history and lifestyle. For example, other diseases that the patient has, might have, or has had can effect the choice of the panel of biomarkers to be analyzed. Drugs that the patient has in his/her system may also affect biomarker selection.

Threshold values for any particular biomarker and associated disease are determined by reference to literature or standard of care criteria or may be determined empirically. In certain embodiments of the invention, thresholds for use in association with biomarkers of the invention are based upon positive and negative predictive values associated with threshold levels of the marker. There are numerous methods for determining thresholds for use in the invention, including reference to standard values in the literature or associated standards of care. The precise thresholds chosen are immaterial as long as they have the desired association with diagnostic output.

The invention is applicable to diagnosis and monitoring of any disease, either in symptomatic or asymptomatic patient populations. For example, the invention can be used for diagnosis of infectious diseases, inherited diseases, and other conditions, such as disease or damage caused by drug or alcohol abuse. The invention can also be applied to assess therapeutic efficacy, potential for disease recurrence or spread (e.g. metastasis).

Methods of the invention can be used on patients known to have a disease, or can be used to screen healthy subjects on a periodic basis. Screening can be done on a regular basis (e.g., weekly, monthly, annually, or other time interval); or as a one time event. The outcome of the analysis may be used to alter the frequency and/or type of screening, diagnostic and/or treatment protocols. Different conditions can be screened for at different time intervals and as a function of different risk factors (e.g., age, weight, gender, history of smoking, family history, genetic risks, exposure to toxins and/or carcinogens etc., or a combination thereof). The particular screening regimen and choice of markers used in connection with the invention are determined at the discretion of the physician or technician.

Biomarkers associated with diseases are shown for example in Shuber (U.S. patent application number 2009/0075266), the content of which is incorporated by reference herein in its entirety. The invention is especially useful in screening for cancer. Examples of biomarkers associated with cancer include FGFR3, matrix metalloproteinase (MMP), neutrophil gelatinase-associated lipocalin (NGAL), MMP/NGAL complex, thymosin β15, thymosin β16, collagen like gene (CLG) product, prohibitin, glutathione-S-transferase, beta-5-tubulin, ubiquitin, tropomyosin, Cyr61, cystatin B, chaperonin 10, and profilin. Examples of MMPs include, but are not limited to, MMP-2, MMP-9, MMP9/NGAL complex, MMP/TIMP complex, MMP/TIMP1 complex, ADAMTS-7 or ADAM-12, among others.

Biomarkers associated with development of breast cancer are shown in Erlander et al. (U.S. Pat. No. 7,504,214), Dai et al. (U.S. Pat. No. 7,514, 209 and 7,171,311), Baker et al. (U.S. Pat. No. 7,056,674 and U.S. Pat. No. 7,081,340), Erlander et al. (US 2009/0092973). The contents of the patent application and each of these patents are incorporated by reference herein in their entirety. Exemplary biomarkers that have been associated with breast cancer include: ErbB2 (Her2); ESR1; BRCA1; BRCA2; p53; mdm2; cyclin1; p27; B_Catenin; BAG1; BIN1; BUB1; C20_orf1; CCNB1; CCNE2; CDC20; CDH1; CEGP1; CIAP1; cMYC; CTSL2; DKFZp586M07; DRS; EpCAM; EstR1; FOXM1; GRB7; GSTM1; GSTM3; HER2; HNRPAB; ID1; IGF1R; ITGA7; Ki_{—}67; KNSL2; LMNB1; MCM2; MELK; MMP12; MMP9; MYBL2; NEK2; NME1; NPD009; PCNA; PR; PREP; PTTG1; RPLPO; Src; STK15; STMY3; SURV; TFRC; TOP2A; and TS.

Biomarkers associated with development of cervical cancer are shown in Patel (U.S. Pat. No. 7,300,765), Pardee et al. (U.S. Pat. No. 7,153,700), Kim (U.S. Pat. No. 6,905,844), Roberts et al. (U.S. Pat. No. 6,316,208), Schlegel (US 2008/0113340), Kwok et al. (US 2008/0044828), Fisher et al. (US 2005/0260566), Sastry et al. (US 2005/0048467), Lai (US 2008/0311570) and Van Der Zee et al. (US 2009/0023137). The contents of each of the articles, patents, and patent applications are incorporated by reference herein in their entirety. Exemplary biomarkers that have been associated with cervical cancer include: SC6; SIX1; human cervical cancer 2 protooncogene (HCCR-2); p27; virus oncogene E6; virus oncogene E7; p16^{INK4A}; Mcm proteins (such as Mcm5); Cdc proteins; topoisomerase 2 alpha; PCNA; Ki-67; Cyclin E; p-53; PAI1; DAP-kinase; ESR1; APC; TIMP-3; RAR-β; CALCA; TSLC1; TIMP-2; DcR1; CUDR; DcR2; BRCA1; p15; MSH2; Rassf1A; MLH1; MGMT; SOX1; PAX1; LMX1A; NKX6-1; WT1; ONECUT1; SPAG9; and Rb (retinoblastoma) proteins.

Biomarkers associated with development of vaginal cancer are shown in Giordano (U.S. Pat. No. 5,840,506), Kruk (US 2008/0009005), Hellman et al. (Br J Cancer. 100(8):1303-1314, 2009). The contents of each of the articles, patents, and patent applications are incorporated by reference herein in their entirety. Exemplary biomarkers that have been associated with vaginal cancer include: pRb2/p130 and Bcl-2.

Biomarkers associated with development of brain cancers (e.g., glioma, cerebellum, medulloblastoma, astrocytoma, ependymoma, glioblastoma) are shown in D'Andrea (US 2009/0081237), Murphy et al. (US 2006/0269558), Gibson et al. (US 2006/0281089), and Zetter et al. (US 2006/0160762). The contents of each of the articles and patent applications are incorporated by reference herein in their entirety. Exemplary biomarkers that have been associated with brain cancers include: epidermal growth factor receptor (EGFR); phosphorylated PKB/Akt; EGFRvIII; FANCI; Nr-CAM; antizyme inhibitor (AZI); BNIP3; and miRNA-21.

Biomarkers associated with development of renal cancer are shown in Patel (U.S. Pat. No. 7,300,765), Soyupak et al. (U.S. Pat. No. 7,482,129), Sahin et al. (U.S. Pat. No. 7,527,933 ), Price et al. (U.S. Pat. No. 7,229,770), Raitano (U.S. Pat. No. 7,507,541), and Becker et al. (US 2007/0292869). The contents of each of the articles, patents, and patent applications are incorporated by reference herein in their entirety. Exemplary biomarkers that have been associated with renal cancers include: SC6; 36P6D5; IMP3; serum amyloid alpha; YKL-40; SC6; and carbonic anhydrase IX (CA IX).

Biomarkers associated with development of hepatic cancers (e.g., hepatocellular carcinoma) are shown in Horne et al. (U.S. Pat. No. 6,974,667), Yuan et al. (U.S. Pat. No. 6,897,018), Hanausek-Walaszek et al. (U.S. Pat. No. 5,310,653), and Liew et al. (US 2005/0152908). The contents of each of the articles, patents, and patent applications are incorporated by reference herein in their entirety. Exemplary biomarkers that have been associated with hepatic cancers include: Tetraspan NET-6 protein; collagen, type V, alpha; glypican 3; pituitary tumor-transforming gene 1 (PTTG1); Galectin 3; solute carrier family 2, member 3, or glucose transporter 3 (GLUT3); metallothionein 1L; CYP2A6; claudin 4; serine protease inhibitor, Kazal type I (SPINK1); DLC-1; AFP; HSP70; CAP2; glypican 3; glutamine synthetase; AFP; AST and CEA.

Biomarkers associated with development of gastric, gastrointestinal, and/or esophageal cancers are shown in Chang et al. (U.S. Pat. No. 7,507,532), Bae et al. (U.S. Pat. No. 7,368,255), Muramatsu et al. (U.S. Pat. No. 7,090,983), Sahin et al. (U.S. Pat. No. 7,527,933), Chow et al. (US 2008/0138806), Waldman et al. (US 2005/0100895), Goldenring (US 2008/0057514), An et al. (US 2007/0259368), Guilford et al. (US 2007/0184439), Wirtz et al. (US 2004/0018525), Filella et al. (Acta Oncol. 33(7):747-751, 1994), Waldman et al. (U.S. Pat. No. 6,767,704), and Lipkin et al. (Cancer Research, 48:235-245, 1988). The contents of each of the articles, patents, and patent applications are incorporated by reference herein in their entirety. Exemplary biomarkers that have been associated with gastric, gastrointestinal, and/or esophageal cancers include: MH15 (Hn1L); RUNX3; midkine; Chromogranin A (CHGA); Thy-1 cell surface antigen (THY1); IPO-38; CEA; CA 19.9; GroES; TAG-72; TGM3; HE4; LGALS3; IL1RN; TRIP13; FIGNL1; CRIP1; S100A4; EXOSC8; EXPI; CRCA-1; BRRN1; NELF; EREG; TMEM40; TMEM109; and guanylin cyclase C.

Biomarkers associated with development of ovarian cancer are shown in Podust et al. (U.S. Pat. No. 7,510,842), Wang (U.S. Pat. No. 7,348,142), O'Brien et al. (U.S. Pat. Nos. 7,291,462, 6,942,978, 6,316,213, 6,294,344, and 6,268,165), Ganetta (U.S. Pat. No. 7,078,180), Malinowski et al. (US 2009/0087849), Beyer et al. (US 2009/0081685), Fischer et al. (US 2009/0075307), Mansfield et al. (US 2009/0004687), Livingston et al. (US 2008/0286199), Farias-Eisner et al. (US 2008/0038754), Ahmed et al. (US 2007/0053896), Giordano (U.S. Pat. No. 5,840,506), and Tchagang et al. (Mol Cancer Ther, 7:27-37, 2008). The contents of each of the articles, patents, and patent applications are incorporated by reference herein in their entirety. Exemplary biomarkers that have been associated with ovarian cancer include: hepcidin; tumor antigen-derived gene (TADG-15); TADG-12; TADG-14; ZEB; PUMP-1; stratum corneum chymotrytic enzyme (SCCE); NES-1; μPA; PAI-2; cathepsin B; cathepsin L; ERCC5; MMP-2; pRb2/p130 gene; matrix metalloproteinase-7 (MMP-7); progesterone-associated endometrial protein (PALP); cancer antigen 125 (CA125); CTAP3; human epididymis 4 (HL4); plasminogen activator urokinase receptor (PLAUR); MUC-1; FGF-2; cSHMT; Tbx3; utrophin; SLPI; osteopontin (SSP1); mesothelin (MSLN); SPON1; interleukin-7; folate receptor 1; and claudin 3.

Biomarkers associated with development of head-and-neck and thyroid cancers are shown in Sidransky et al. (U.S. Pat. No. 7,378,233), Skolnick et al. (U.S. Pat. No. 5,989,815), Budiman et al. (US 2009/0075265), Hasina et al. (Cancer Research, 63:555-559, 2003), Kebebew et al. (US 2008/0280302), and Ralhan (Mol Cell Proteomics, 7(6):1162-1173, 2008). The contents of each of the articles, patents, and patent applications are incorporated by reference herein in their entirety. Exemplary biomarkers that have been associated with head-and-neck and thyroid cancers include: BRAF; Multiple Tumor Suppressor (MTS); PAI-2; stratifin; YWHAZ; S100-A2; S100-A7 (psoriasin); S100-A11 (calgizarrin); prothymosin alpha (PTHA); L-lactate dehydrogenase A chain; glutathione S-transferase Pi; APC-binding protein EB1; fascin; peroxiredoxin2; carbonic anhydrase I; flavin reductase; histone H3; ECM1; TMPRSS4; ANGPT2; T1MP1; LOXL4; p53; IL-6; EGFR; Ku70; GST-pi; and polybromo-1D.

Biomarkers associated with development of colorectal cancers are shown in Raitano et al. (U.S. Pat. No. 7,507,541), Reinhard et al. (U.S. Pat. No. 7,501,244), Waldman et al. (U.S. Pat. No. 7,479,376); Schleyer et al. (U.S. Pat. No. 7,198,899); Reed (U.S. Pat. No. 7,163,801), Robbins et al. (U.S. Pat. No. 7,022,472), Mack et al. (U.S. Pat. No. 6,682,890), Tabiti et al. (U.S. Pat. No. 5,888,746), Budiman et al. (US 2009/0098542), Karl (US 2009/0075311), Arjol et al. (US 2008/0286801), Lee et al. (US 2008/0206756), Mori et al. (US 2008/0081333), Wang et al. (US 2008/0058432), Belacel et al. (US 2008/0050723), Stedronsky et al. (US 2008/0020940), An et al. (US 2006/0234254), Eveleigh et al. (US 2004/0146921), and Yeatman et al. (US 2006/0195269). The contents of each of the articles, patents, and patent applications are incorporated by reference herein in their entirety. Exemplary biomarkers that have been associated with colorectal cancers include: 36P6D5; TTK; CDX2; NRG4; TUCAN; hMLH1; hMSH2; M2-PK; CGA7; CJA8; PTP.alpha.; APC; p53; Ki-ras; complement C3a des-arg; alpha1-antitrypsin; transferrin; MMP-11; CA-19-9; TPA; TPS; TIMP-1; C10orf3; carcinoembryonic antigen (CEA); a soluble fragment of cytokeratin 19 (CYFRA 21-1); TAC1; carbohydrate antigen 724 (CA72-4); nicotinamide N-methyltransferase (NNMT); pyrroline-5-carboxylate reductase (PROC); S-adenosylhomocysteine hydrolase (SAHH); IBABP-L polypeptide; and Septin 9.

Biomarkers associated with development of prostate cancer are shown in Sidransky (U.S. Pat. No. 7,524,633), Platica (U.S. Pat. No. 7,510,707), Salceda et al. (U.S. Pat. No. 7,432,064 and U.S. Pat. No. 7,364,862), Siegler et al. (U.S. Pat. No. 7,361,474), Wang (U.S. Pat. No. 7,348,142), Ali et al. (U.S. Pat. No. 7,326,529), Price et al. (U.S. Pat. No. 7,229,770), O'Brien et al. (U.S. Pat. No. 7,291,462), Golub et al. (U.S. Pat. No. 6,949,342), Ogden et al. (U.S. Pat. No. 6,841,350), An et al. (U.S. Pat. No. 6,171,796), Bergan et al. (US 2009/0124569), Bhowmick (US 2009/0017463), Srivastava et al. (US 2008/0269157), Chinnaiyan et al. (US 2008/0222741), Thaxton et al. (US 2008/0181850), Dahary et al. (US 2008/0014590), Diamandis et al. (US 2006/0269971), Rubin et al. (US 2006/0234259), Einstein et al. (US 2006/0115821), Paris et al. (US 2006/0110759), Condon-Cardo (US 2004/0053247), and Ritchie et al. (US 2009/0127454). The contents of each of the articles, patents, and patent applications are incorporated by reference herein in their entirety. Exemplary biomarkers that have been associated with prostate cancer include: PSA; GSTP1; PAR; CSG; MIF; TADG-15; p53; YKL-40; ZEB; HOXC6; Pax 2; prostate-specific transglutaminase; cytokeratin 15; MEK4; MIP1-β; fractalkine; IL-15; ERGS; EZH2; EPC1; EPC2; NLGN-4Y; kallikrein 11; ABP280 (FLNA); AMACR; AR; BM28; BUB3; CaMKK; CASPASE3; CDK7; DYNAMIN; E2F1; E-CADHERIN; EXPORTIN; EZH2; FAS; GAS7; GS28; ICBP90; ITGA5; JAGGED1; JAM1; KANADAPTIN; KLF6; KRIP1; LAP2; MCAM; MIB1 (MKI67); MTA1; MUC1; MYOSIN-VI; P27; P63; P27; PAXILLIN; PLCLN; PSA(KLK3); RAB27; RBBP; RIN1; SAPKα; TPD52; XIAP; ZAG; and semenogelin II.

Biomarkers associated with development of pancreatic cancer are shown in Sahin et al. (U.S. Pat. No. 7,527,933), Rataino et al. (U.S. Pat. No. 7,507,541), Schleyer et al. (U.S. Pat. No. 7,476,506), Domon et al. (U.S. Pat. No. 7,473,531), McCaffey et al. (U.S. Pat. No. 7,358,231), Price et al. (U.S. Pat. No. 7,229,770), Chan et al. (US 2005/0095611), Mitchl et al. (US 2006/0258841), and Faca et al. (PLoS Med 5(6):e123, 2008). The contents of each of the articles, patents, and patent applications are incorporated by reference herein in their entirety. Exemplary biomarkers that have been associated with pancreatic cancer include: CA19.9; 36P6D5; NRG4; ASCT2; CCR7; 3C4-Ag; KLK11; Fibrinogen γ; and YKL40.

Biomarkers associated with development of lung cancer are shown in Sahin et al. (U.S. Pat. No. 7,527,933), Hutteman (U.S. Pat. No. 7,473,530), Bae et al. (U.S. Pat. No. 7,368,255), Wang (U.S. Pat. No. 7,348,142), Nacht et al. (U.S. Pat. No. 7,332,590), Gure et al. (U.S. Pat. No. 7,314,721), Patel (U.S. Pat. No. 7,300,765), Price et al. (U.S. Pat. No. 7,229,770), O'Brien et al. (U.S. Pat. No. 7,291,462 and U.S. Pat. No. 6,316,213), Muramatsu et al. (U.S. Pat. No. 7,090,983), Carson et al. (U.S. Pat. No. 6,576,420), Giordano (U.S. Pat. No. 5,840,506), Guo (US 2009/0062144), Tsao et al. (US 2008/0176236), Nakamura et al. (US 2008/0050378), Raponi et al. (US 2006/0252057), Yip et al. (US 2006/0223127), Pollock et al. (US 2006/0046257), Moon et al. (US 2003/0224509), and Budiman et al. (US 2009/0098543). The contents of each of the articles, patents, and patent applications are incorporated by reference herein in their entirety. Exemplary biomarkers that have been associated with lung cancer include: COX-2; COX4-2; RUNX3; aldoketoreductase family 1, member B 10; peroxiredoxin 1 (PRDX1); TNF receptor superfamily member 18; small proline-rich protein 3 (SPRR3); SOX1; SC6; TADG-15; YKL40; midkine; DAP-kinase; HOXA9; SCCE; STX1A; HIF1A; CCT3; HLA-DPB1; MAFK; RNF5; KIF11; GHSR1b; NTSR1; FOXM1; and PUMP-1.

Biomarkers associated with development of skin cancer (e.g., basal cell carcinoma, squamous cell carcinoma, and melanoma) are shown in Roberts et al. (U.S. Pat. No. 6,316,208), Polsky (U.S. Pat. No. 7,442,507), Price et al. (U.S. Pat. No. 7,229,770), Genetta (U.S. Pat. No. 7,078,180), Carson et al. (U.S. Pat. No. 6,576,420), Moses et al. (US 2008/0286811), Moses et al. (US 2008/0268473), Dooley et al. (US 2003/0232356), Chang et al. (US 2008/0274908), Alani et al. (US 2008/0118462), Wang (US 2007/0154889), and Zetter et al. (US 2008/0064047). The contents of each of the articles, patents, and patent applications are incorporated by reference herein in their entirety. Exemplary biomarkers that have been associated with skin cancer include: p27; Cyr61; ADAMTS-7; Cystatin B; Chaperonin 10; Profilin; BRAF; YKL-40; DDX48; erbB3-binding protein; biliverdin reductase; PLAB; L1CAM; SAA; CRP; SOX9; MMP2; CD 10; and ZEB.

Biomarkers associated with development of multiple myeloma are shown in Coignet (U.S. Pat. No. 7,449,303), Shaughnessy et al. (U.S. Pat. No. 7,308,364), Seshi (U.S. Pat. No. 7,049,072), and Shaughnessy et al. (US 2008/0293578, US 2008/0234139, and US 2008/0234138). The contents of each of the articles, patents, and patent applications are incorporated by reference herein in their entirety. Exemplary biomarkers that have been associated with multiple myeloma include: JAG2; CCND1; MAF; MAFB; MMSET; CST6; RAB7L1; MAP4K3; HRASLS2; TRAIL; IG; FGL2; GNG11; MCM2; FLJ10709; TRIM13; NADSYN1; TRIM22; AGRN; CENTD2; SESN1; TM7SF2; NICKAP1; COPG; STAT3; ALOX5; APP; ABCB9; GAA; CEP55; BRCA1; ANLN; PYGL; CCNE2; ASPM; SUV39H2; CDC25A; IFIT5; ANKRA2; PHLDB1; TUBA1A; CDCA7; CDCA2; HFE; RIF1; NEIL3; SLC4A7; FXYD5; MCC; MKNK2; KLHL24; DLC1; OPN3; B3GALNT1; SPRED1; ARHGAP25; RTN2; WNT16; DEPDC1; STT3B; ECHDC2; ENPP4; SAT2; SLAMF7; MAN1C1; INTS7; ZNF600; L3MBTL4; LAPTM4B; OSBPL10; KCNS3; THEX1. CYB5D2; UNC93B1; SIDT1; TMEM57; HIGD24; FKSG44; C14orf28; LOC387763; TncRNA; C18orf1; DCUN1D4; FANCI; ZMAT3; NOTCH1; BTG2; RAB1A; TNFRSF10B; HDLBP; RIT1; KIF2C; S100A4; MEIS1; SGOL2; CD302; COX2; C5orf34; FAM111B; C18orf54; and TP53.

Biomarkers associated with development of leukemia are shown in Ando et al. (U.S. Pat. No. 7,479,371), Coignet (U.S. Pat. No. 7,479,370 and U.S. Pat. No. 7,449,303), Davi et al. (U.S. Pat. No. 7,416,851), Chiorazzi (U.S. Pat. No. 7,316,906), Seshi (U.S. Pat. No. 7,049,072), Van Baren et al. (U.S. Pat. No. 6,130,052), Taniguchi (U.S. Pat. No. 5,643,729), Insel et al. (US 2009/0131353), and Van Bockstaele et al. (Blood Rev. 23(1):25-47, 2009). The contents of each of the articles, patents, and patent applications are incorporated by reference herein in their entirety. Exemplary biomarkers that have been associated with leukemia include: SCGF; JAG2; LPL; ADAM29; PDE; Cryptochrome-1; CD49d; ZAP-70; PRAME; WT1; CD15; CD33; and CD38.

Biomarkers associated with development of lymphoma are shown in Ando et al. (U.S. Pat. No. 7,479,371), Levy et al. (U.S. Pat. No. 7,332,280), and Arnold (U.S. Pat. No. 5,858,655). The contents of each of the articles, patents, and patent applications are incorporated by reference herein in their entirety. Exemplary biomarkers that have been associated with lymphoma include: SCGF; LMO2; BCL6; FN1; CCND2; SCYA3; BCL2; CD79a; CD7; CD25; CD45RO; CD45RA; and PRAD1 cyclin.

Biomarkers associated with development of bladder cancer are shown in Price et al. (U.S. Pat. No. 7,229,770), Orntoft (U.S. Pat. No. 6,936,417), Haak-Frendscho et al. (U.S. Pat. No. 6,008,003), Feinstein et al. (U.S. Pat. No. 6,998,232), Elting et al. (US 2008/0311604), and Wewer et al. (2009/0029372). The contents of each of the patent applications and each of these patents are incorporated by reference herein in their entirety. Exemplary biomarkers that have been associated with bladder cancer include: NT-3; NGF; GDNF; YKL-40; p53; pRB; p21; p27; cyclin E1; Ki67; Fas; urothelial carcinoma-associated 1; human chorionic gonadotropin beta type II; insulin-like growth factor-binding protein 7; sorting nexin 16; chondroitin sulfate proteoglycan 6; cathepsin D; chromodomain helicase DNA-binding protein 2; nell-like 2; tumor necrosis factor receptor superfamily member 7; cytokeratin 18 (CK18); ADAMS; ADAM10; ADAM12; MMP-2; MMP-9; KAI1; and bladder tumor fibronectin (BTF).

Any combination of biomarkers may be used with methods of the invention. In certain embodiments, the biomarkers are nucleic acid biomarkers. In other embodiments, the biomarkers are protein biomarkers. In still other embodiments, a combination of nucleic acid and protein biomarkers are applied. When using a combination of nucleic acid and protein biomarkers, certain embodiments of the methods are performed such that nucleic acid biomarkers are assayed before protein biomarkers are assayed.

Nucleic acid biomarkers generally produce a binary result, i.e., presence or absence of a mutation. Protein biomarkers are generally considered quantitative biomarkers for which a level or amount of the biomarker present in comparison to a reference level or amount indicates a clinical status. As already discussed herein, threshold values for any particular biomarker and associated disease may be determined by reference to literature or standard of care criteria or may be determined empirically.

Biomarkers may be assayed by any method known in the art. For example, mutations in nucleic acid biomarkers may be detected by using labeled probes or by sequencing technology, such as single molecule sequencing or Sanger sequencing. Single molecule sequencing by synthesis is shown in, for example, Harris (U.S. Pat. No. 7,282,337) and Quake (US 2002/0164629), the content of each of which is incorporated by reference herein in its entirety. Protein biomarkers may be assayed by, for example, ELISA or Western Blot analysis.

Another aspect of the invention provides methods that iteratively analyze sets of biomarkers. By including multiple biomarkers at each step, the diagnostic power and accuracy of the result is increased. Those methods of the invention provide for diagnosing a disease including obtaining a sample from a subject, conducting a first set of assays on a first set of biomarkers in the sample, assigning a binary output for each of the biomarkers in the first set based upon a standard-of-care threshold for disease diagnosis for each biomarker in the first set, aggregating the outputs into a first single output score, in which the first score is indicative of a positive or a negative diagnosis of a disease, conducting a second set of assays on a second set of biomarkers in the sample if the first single output score is indicative of a negative diagnosis of the disease, assigning a binary output for each of the biomarkers in the second set based upon a standard-of-care threshold for disease diagnosis for each biomarker in the second set, and aggregating the outputs into a second single output score, in which the second score is indicative of a positive or a negative diagnosis of the disease.

Based on the result of the second set of biomarkers, methods of the invention further include conducting at least one additional set of assays on the sample, in which the additional sets of assays are conducted serially and each set of assays includes a different set of biomarkers. The assays are conducted until an aggregated single output score with respect to a set of biomarkers is obtained that is a positive result for the disease to be diagnosed.

Those embodiments of the methods of the invention allow multiplex analysis of a plurality of biomarkers in order to increase the diagnostic power and accuracy of the result. The results from each set of biomarkers, are normalized and a diagnostic score is produced based upon the normalized biomarker data. In certain embodiments, each biomarker is assigned a binary result (e.g., 1/0 or yes/no) based upon whether a mutation is detected in the biomarker or whether the detected level of the biomarker in the patient sample exceeds or is lower than a predetermined threshold. Then, a cumulative score is obtained by adding the binary results in order to produce a diagnostic score. The diagnostic score determines whether further assays need to be conducted. For example, if the majority of biomarkers in the first set produce a negative result, then a second set of biomarkers should be evaluated. In other embodiments, if only a single biomarker in the first set produces a negative result, then a second set of biomarkers should be evaluated. In another embodiment, biomarker results are weighted based upon known diagnostic criteria and/or patient history, lifestyle, symptoms, and the like. The resulting aggregate weighted score is used for clinical assessment.

In certain embodiments of the invention, the readout of the plurality of biomarkers need not be binary. Rather, the readout may take into consideration the predictive value of each of the biomarkers for the condition being assessed. This is a form of weighting based upon known risk factors, diagnostic criteria, and patient history and can be tuned to reflect the degree of confidence that one expects from the assay. Methods of the invention allow the generation of a signature based upon results obtained from a plurality of biomarkers, wherein the signature is indicative of the presence/absence of disease, the stage of disease, or prognostic factors (such as likelihood of recurrence, assessment of response to treatment, and risk of developing disease).

Incorporation by Reference

References and citations to other documents, such as patents, patent applications, patent publications, journals, books, papers, web contents, have been made throughout this disclosure. All such documents are hereby incorporated herein by reference in their entirety for all purposes.

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

### EXAMPLES

### Example 1

### Serial Analysis of Biomarkers for Bladder Cancer Diagnosis

A cohort of samples from bladder cancer recurrence studies were tested using methods of the invention in order to maximize sensitivity and NPV. The nucleic acid marker FGFR3 was first assayed to determine whether there existed a mutation in the FGFR3 sequence that had a known link to bladder cancer. Samples that were negative for the FGFR3 mutation were then assayed for the protein marker ADAM12.

The ADAM12 marker is currently assessed by western analysis, and the diagnostic criteria applied was presence or absence of ADAM12. Samples that were negative for ADAM12 were then assayed for MMP-9, and those that were negative were for MMP-9 were then assayed for MMP-2.

Table 1 below provides clinical performance data that shows that as one, two, three or four markers were applied, sensitivity and NPV of the method was maximized. Because protein results (as determined by ELISA) are quantitative, different cutoffs were chosen based on literature regarding the above protein biomarkers. Moving the cutoff of any one protein marker at a time may be used as a rheostat to increase or decrease sensitivity and specificity. Data in Table 1 show that one MMP-9 cutoff was used and two different MMP-2 cutoffs were used. Samples above either cutoff were considered positive while samples below were considered negative in each case. Data show that as the MMP-2 cutoff was shifted, the sensitivity, specificity, and the NPV of the assay changed.

Data herein demonstrate that methods of the invention maximize sensitivity and NPV, thus eliminating as many ambiguous results as possible. Methods of the invention thereby limit the number of patients who must endure unnecessary procedures and optimize identification of patients who would certainly benefit from continual monitoring and/or intervention.

TABLE 1

| | | | | | Cancers |
|---|---|---|---|---|---|
| Biomarker | Cutoff | Sensitivity | Specificity | NPV | Missed |
| FGFR3 | | 44% | 80% | 90% | 14 |
| | | [24-65%] | [73-86%] | [84-94%] | |
| FGFR3 + | Adam12 < 1 | 76% | 60% | 94% | 6 |
| ADAM12 | | [55-91%] | [52-68%] | [87-98%] | |
| FGFR3 + | MMP-0 < .517 | 92% | 36% | 97% | 2 |
| ADAM12 + | | [74-99%] | 28-44%] | [88-100%] | |
| MMP-9 | | | | | |
| FGFR3 + | MMP-0 < .517 | 96% | 34% | 98% | 1 |
| ADAM12 + | MMP-2 < .900 | [80-100%] | [27-42%] | [90-100%] | |
| MMP-9 + | | | | | |
| MMP-2 | | | | | |
| FGFR3 + | MMP-0 < .517 | 100% | 27% | 100% | 0 |
| ADAM12 + | MMP-2 < .413 | [86-100%] | [20-35%] | [92-100%] | |
| MMP-9 + | | | | | |
| MMP-2 | | | | | |

What is claimed is:

1. A method for diagnosing a disease state, the method comprising:
obtaining a sample from a subject;
conducting a first assay to determine whether a first biomarker is positive or negative for a disease; and
conducting a second assay to determine whether a second biomarker is positive or negative for the disease if the first assay produced a negative result.

2. The method according to claim 1, further comprising conducting at least one additional assay on the sample, wherein the additional assays are conducted serially and each assay is conducted on a different biomarker.

3. The method according to claim 2, wherein the assays are conducted until a positive result with respect to a biomarker indicative of the disease is detected.

4. The method according to claim 1, wherein the first biomarker is a nucleic acid biomarker.

5. The method according to claim 4, wherein the first assay detects presence or absence of a mutation, wherein presence of the mutation is indicative of a positive result for the disease.

6. The method according to claim 1, wherein the second biomarker is a protein biomarker.

7. The method according to claim 6, wherein the second assay measures a level of the protein in the sample.

8. The method according to claim 7, wherein a level exceeding a predetermined threshold for the protein is indicative of a positive result for the disease.

9. The method according to claim 7, wherein a level below a predetermined threshold for the protein is indicative of a positive result for the disease.

10. The method according to claim 1, wherein the first and second markers have a known standard-of-care threshold for disease diagnosis.

11. A method for diagnosing a disease, the method comprising:
obtaining a sample from a subject;
conducting a first set of assays on a first set of biomarkers;
assigning a value for each of the biomarkers in the first set based upon a standard-of-care threshold for disease diagnosis for each biomarker in the first set;
aggregating the values into a first single output score, wherein the first score is indicative of a positive or a negative diagnosis of a disease;
conducting a second set of assays on a second set of biomarkers if the first single output score is indicative of a negative diagnosis of the disease;
assigning a value for each of the biomarkers in the second set based upon a standard-of-care threshold for disease diagnosis for each biomarker in the second set; and
aggregating the values into a second single output score, wherein the second score is indicative of a positive or a negative diagnosis of the disease.

12. The method according to claim 11, further comprising conducting at least one additional set of assays on the sample, wherein the additional sets of assays are conducted serially and each set of assays comprises a different set of biomarkers.

13. The method according to claim 12, wherein the assays are conducted until an aggregated single output score with respect to a set of biomarkers is obtained that is a positive result for the disease to be diagnosed.

14. The method according to claim 11, wherein the value for each of the biomarkers in the first set and the value for each of the biomarkers in the second set are binary values.

15. The method according to claim 14, wherein the first set of biomarkers are nucleic acid biomarkers.

16. The method according to claim 15, wherein the first set of assays detects presence or absence of a mutation for each biomarker in the first set.

17. The method according to claim 16, wherein presence of a mutation for a biomarker in the first set is assigned an output of “1”, and absence of a mutation for a biomarker in the first set is assigned an output of “0”.

18. The method according to claim 14, wherein the second set of biomarkers are protein biomarkers.

19. The method according to claim 18, wherein the second set of assays measure levels of the proteins in the sample.

20. The method according to claim 19, wherein a level exceeding a predetermined threshold for each protein in the second set is assigned an output of “1”, and a level below a predetermined threshold for each protein in the second set is assigned an output of “0”.

21. The method according to claim 19, wherein a level below a predetermined threshold for each protein in the second set is assigned an output of “1”, and a level exceeding a predetermined threshold for each protein in the second set is assigned an output of “0”.

22. A method for diagnosing a disease, the method comprising:
obtaining a sample from a subject; and
conducting a plurality of assays on the sample, wherein the assays are conducted serially and each assay is conducted on a different biomarker, wherein a further assay is conducted only if a previously conducted assay produced a negative result with respect to the biomarker being assayed.

23. The method according to claim 22, wherein the assays are conducted until a positive result with respect to a biomarker indicative of the disease is detected.

24. The method according to claim 22, wherein nucleic acid biomarkers are assayed before protein biomarkers.

25. The method according to claim 22, wherein the biomarkers have a known standard-of-care threshold for disease diagnosis.
